(19) United States
(12) Patent Application Publication
(10) Pub. No.: US 2015/0037867 A1
(43) Pub. Date: Feb. 5, 2015

(54) PURIFICATION METHOD

(71) Applicant: Castech Laboratories, LLC, Encinitas, CA (US)

(72) Inventor: Jacek C. Ostrowski, Encinitas, CA (US)

(21) Appl. No.: 14/379,440

(22) PCT Filed: Feb. 15, 2013
(86) PCT No.: PCT/US13/26483

§ 371 Date: Aug. 18, 2014

Related U.S. Application Data
(60) Provisional application No. 61/600,370, filed on Feb. 17, 2012.
(60) Provisional application No. 61/607,902, filed on Mar. 7, 2012.
(60) Provisional application No. 61/611,269, filed on Mar. 15, 2012.
(60) Provisional application No. 61/611,262, filed on Mar. 15, 2012.

(51) Int. Cl. C07K 1/14; C07K 16/00; C12N 9/00

(52) U.S. Cl. CPC C07K 1/14; C12N 9/00; C07K 16/00
USPC 435/183; 530/415; 530/387.1

(57) ABSTRACT

The invention provides methods for purifying protein products.

### CROSS REFERENCE TO RELATED APPLICATIONS

This application claims the benefit of priority of U.S. provisional application Ser. No. 61/600,370, filed Feb. 17, 2012; U.S. provisional application Ser. No. 61/607,902, filed Mar. 7, 2012; U.S. provisional application Ser. No. 61/611,262, filed Mar. 15, 2012; and U.S. provisional application Ser. No. 61/611,269, filed Mar. 15, 2012, which applications are herein incorporated by reference.

### BACKGROUND

Proteins are a major drug class and are widely utilized in medicine. Unfortunately, proteins that are grown in bacterial culture require purification to separate them from other bacteria-derived proteins and impurities (e.g. endotoxins). Removal of oxidizable impurities (e.g. endotoxins, peptidoglycans, teichoic acid, and lipoteichoic acid) is important for the safety of the resulting proteins, since oxidizable impurities can cause severe reactions, which can lead to death. Certain existing methods to remove endotoxin from protein solutions are based on preferentially binding (non-covalently) oxidizable impurities to a stationary phase while allowing the protein to flow through (Endotoxins: Structure, Function and Recognition, Xiaoyuan Wang and Peter J. Quinn, Springer, Dordrecht Heidelberg London New York, 2010; and Petsch D. and Anspach F. B., J. Biotechnology, 76, 2000, 97-119; Hideo Igarashi et al., “Purification and Characterization of Staphylococcus aureus FRI 1169 and 587 Toxic Shock Syndrome Exotoxins”, Infection and Immunity, April 1984, p. 175-181; Zenker et al. “Characterization of Peptiodoglycan Trimers after Gel Chromatography and Reversed-phase HPLC by Positive-ion Desorption Mass Spectrometry” Rapid Communications in Mass Spectrometry, vol. 10, 1956-1960 (1996)) The main flaw of these methods is an incomplete removal of oxidizable impurities, since not all of the oxidizable impurities become bound, and thus are not removed.

In another purification method, substrates are generated that bind only proteins and the oxidizable impurity is eluted away. The protein is then eluted from the substrate. This method also poses a risk of incomplete removal of the oxidizable impurity and a potential loss of protein if the amount of protein exceeds the binding capacity of the substrate.

Accordingly, there is currently a need for improved methods for separating proteins from oxidizable impurities. The improved method should: remove a high percentage of oxidizable impurities from protein solutions, cause little or no loss of proteins, cause little or no change in the proteins, be inexpensive, and/or be viable on a commercially useful scale.

### SUMMARY

An improved method for separating proteins from oxidizable impurities has been discovered. The improved method typically removes a high percentage of oxidizable impurities from protein solutions, causes little or no loss of proteins, causes little or no change in the proteins, is relatively inexpensive compared to existing methods, and is viable on a commercially useful scale.

Accordingly, in one embodiment the invention provides a method comprising:

contacting a mixture that comprises a protein and one or more oxidizable impurity with an oxidizing agent to provide a resulting mixture that comprises an oxidized impurity; and

separating the protein from the oxidized impurity by contacting the resulting mixture with a material that covalently bonds with the oxidized impurity.

### DETAILED DESCRIPTION

The following definitions are used, unless otherwise described: A (C₁-C₃)alcohol denotes both straight and branched groups; but reference to an individual radical such as propanol embraces only the straight chain radical, a branched chain isomer such as isopropanol being specifically referred to.

The term saccharide includes monosaccharides, disaccharides, trisaccharides and polysaccharides. The term includes glucose, sucrose fructose and ribose, as well as deoxy sugars such as deoxyribose and the like.

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein).

The term “peptide” describes a sequence of 2 to 25 amino acids (e.g. as defined hereinabove) or peptidyl residues. The sequence may be linear or cyclic. For example, a cyclic peptide can be prepared or may result from the formation of disulfide bridges between two cysteine residues in a sequence. A peptide can be linked to the remainder of a compound of formula I through the carboxy terminus, the amino terminus, or through any other convenient point of attachment, such as, for example, through the sulfur of a cysteine. Preferably a peptide comprises 3 to 25, or 5 to 21 amino acids. Peptide derivatives can be prepared as disclosed in U.S. Pat. Nos. 4,612,302; 4,853,371; and 4,684,620, or as described in the Examples hereinbelow. Peptide sequences specifically recited herein are written with the amino terminus on the left and the carboxy terminus on the right.

Proteins include biochemical compounds comprising one or more polypeptides typically folded into a globular or fibrous form, facilitating a biological function. In one embodiment of the invention the protein is an enzyme, an antibody, a structural or mechanical protein (e.g. actin or myosin), a protein used for cell adhesion, a protein used for cell signaling, or a protein utilized in the cell cycle.

The term “protein product” includes peptides and proteins.

### Mixtures

In one embodiment of the invention the mixture comprises one or more solvents. The mixture can comprise any suitable solvents that allow for the oxidation of the oxidizable impurities and the subsequent separation of the oxidized impurities from the protein. For example, the solution can comprises water, a (C₁-C₃)alcohol, DMSO, dioxane, dimethoxy ethane, acetonitrile, and DMF, or a mixture thereof. According to the methods of the invention the oxidation can be carried out in a mixture that comprises one or more solvents and the mixture can be contacted with the material and the proteins washed from the material using the same or different solvents or mixtures of solvents.

In one embodiment the solution in which the oxidation takes place or the solution that is used for separating the protein product from the oxidized impurity may be buffered. Suitable buffers are well known and can be selected by one skilled in the art to be compatible with the protein product and/or reaction conditions employed.

### Oxidizable Impurities

Oxidizable impurities include any unwanted component of a protein containing mixture that can be oxidized to form an oxidized impurity that can be separated from the protein. For example, the term oxidizable impurity includes endotoxins, peptidoglycans, teichoic acids and lipoteichoic acids.

### Endotoxins

Endotoxin includes lipopolysaccharides that constitute the outer leaflet of the outer membrane of most Gram-negative bacteria. Lipopolysaccharide is comprised of a hydrophilic polysaccharide and a hydrophobic component known as lipid A which is responsible for the major bioactivity of endotoxin. The polysaccharide can be comprised of various sugars including galactose, glucose, glucosamine, n-acetalglucoseamine, heptose, and 3-deoxy-D-manno-oct-2-ulopyranosonic acid.

### Peptidoglycans

Peptidoglycans constitute the outer leaflet of the outer membrane of most Gram-negative bacteria. Peptidoglycane is comprised of a polysaccharide and a short peptide. The polysaccharide can be comprised of various sugars including galactose, glucose, glucosamine, n-acetalgiucoseamine, heptose, and 3-deoxy-D-manno-oct-2-ulopyranosonic acid.

### Teichoic and Lipoteichoic Acids

Teichoic and lipoteichoic acids constitute of most Gram-positive bacteria. Teichoic and lipoteichoic acids are comprised of a polysaccharide and for the case of the lipoteichoic acids a lipid component. The polysaccharide can be comprised of various sugars including galactose, glucose, glucosamine, n-acetalglucoseamine, heptose, and 3-deoxy-D-manno-oct-2-ulopyranosonic acid.

### Oxidizing Agents

Any suitable oxidizing agent can be used in the methods of the invention. In one embodiment of the invention the oxidizing agent will oxidize one or more groups on the oxidizable impurity without damaging the protein. In one embodiment of the invention the oxidizing agent is suitable for oxidizing one or more hydroxy groups on the oxidizable impurity to the corresponding aldehydes. Suitable oxidizing agents include periodate salts (e.g. sodium periodate or potassium periodate IBX, Dess-Martin reagent) and lead tetraacetate.

The concentration of oxidizable impurity in solution can be measured prior to treatment with the oxidizing agent to ensure that an adequate amount of oxidizing agent is used. Typically 1 to 20 equivalents of oxidizing agent is used. The extent of oxidation can be monitored using Schiffs reagent.

In one embodiment the solution containing the protein and oxidizable impurity is brought into contact with a solution of a periodate salt (e.g. a sodium or potassium salt), wherein the final periodate concentration is between 1 and 20 equivalent. The periodate salt may be dissolved in water. The reaction can be buffered to a pH of 4.0-8.0 (e.g. between pH 4.5 and 7.2). The time of incubation is typically from 5 minutes to 1 day and usually is between 1 to 2 hours. The incubation is typically protected from light. The temperature of the reaction is typically between 20° C. and 50° C., however, the reaction can be carried out at any suitable temperature. In one embodiment the reaction is carried out at a temperature where the protein is not denatured (e.g. at about 25±5° C.).

### Material

The material that associates with the oxidized impurity can be any material that selectively associates with the oxidized impurity so that it can be separated from the protein. In one embodiment of the invention the material covalently bonds with the oxidized impurity so that it can be separated from the protein.

In one embodiment of the invention, the material comprises a plurality of groups that can covalently bond with the oxidized impurity. When the oxidized impurity comprises one or more aldehyde groups, the material typically comprises a plurality of groups that can react with the aldehyde groups to form covalent bonds. For example, the material can comprise one or more hydrazide groups (e.g. a group of formula —NH—NH₂). Suitable hydrazide groups include semicarbazides, thiosemicarbazides, and aryl hydrazide.

The material can be in any form that is suitable to allow for the separation of the oxidized impurity and the protein. For example, the material can be in the form of a bead, a powder, a gel, a nanoparticle, a fabric, a membrane, and a surface. In one embodiment of the invention material is a bead. In another embodiment the material is a bead that comprises comprises sugar (agarose or dextran), silica, polymer (polyacrylamide), glass, metal (magnetic and non-magnetic), a metal coated silica particle, a silica coated metal particle, a sugar coated metal particle, or a polymer coated metal particle. Typically the bead size may vary from 5 nm to 1000 μm. In one embodiment of the invention the material comprises beads in the range of 10 μm to 170 μm.

Materials that comprise groups capable of associating with the oxidized impurity can be purchased from Fisher Scientific, Biorad, and Calbiochem-Novabiochem. Materials that comprise groups capable of associating with the oxidized impurity (e.g. resins) can also be synthesized using known methods, for example see O'Shannessy D. J. Chromat. A, 105, 1990, 13-21 and the references cited therein. Materials that comprise groups capable of associating with the oxidized impurity also include membranes. For example, membranes with hydrazide functionalities have been reported by Ramani M. P. S and Ramachandhran V. Desalination, 90, 1993, 31.

### Separations

The oxidized impurity and the protein can be separated using any suitable technique. For example, the oxidized impurity and the protein can be separated by passing the solution that comprises the protein and the oxidized impurity through a column that contains the material that associates (e.g. covalently bonds) with the oxidized impurity and eluting the column with a suitable solvent.

The oxidized impurity and the protein can also be separated by contacting the solution that comprises the protein and the oxidized impurity with a stationary phase that associates with (e.g. covalently bonds) the oxidized impurity, and washing the protein from the stationary phase using any suitable means (e.g. using an elution solvent).

According to one embodiment, the invention comprises of a method for removing an oxidizable impurity from a protein solution that is contaminated with an oxidizable impurity. The removal of the oxidizable impurity can be accomplished when oxidized impurity is passed over a column that contains a stationary phase (e.g. a bead-based matrix) that is functionalized with groups that will associate with or bond to the oxidized impurity (e.g. hydrazide groups). The oxidized impurity associates with (e.g. covalently bonds to) the matrix and remains associated (e.g. bound), while the protein flows through. Accordingly, when a solution of protein that was grown in bacteria and that contains oxidizable impurities, is treated as described above and purified over a hydrazide column such impurities are removed.

When the oxidized impurity binds covalently to the material, the methods of the invention may provide superior separations compared to separation methods that rely on electrostatic and/or Van der Waals interactions to bind either impurity or protein to a substrate. When the binding of the impurity to a substrate is electrostatic and/or Van der Waals in nature, impurity can pass through with the protein, providing a less pure product. When separations involve the protein being bound to the substrate electrostatically, some protein can pass through with the impurities, reducing protein yield. Accordingly, the methods of the invention may yield a more pure product when the oxidized impurity binds covalently to the material.

Oxidation of the impurity can yield multiple aldehyde groups on the impurity. This can increase the chances of binding to the material, and may lead to multiple bonds being formed with one impurity and the material. These covalent bonds are not affected by salts and cannot be easily reversed. Once the impurity is bound to the stationary phase it is difficult to remove.

According to one embodiment of the invention the hydrazied stationary phase can be poured into a column. The volume of which depends on the volume of the protein oxidized impurity solution and the initial measured concentration of oxidized impurity. For each measured EU/mL 0.5 mL to 5 mL of beads can typically be used. The oxidized solution containing the oxidized impurity and protein can be added directly with the removal of any unreacted periodate salt. This can be directly flowed over the stationary phase or can be incubated up to two hours after the full addition of protein and oxidized impurity solution. The mobile phase can be buffered to a pH of 5.0-8.0 (e.g. between pH 6.5 and 7.2). The temperature of the column material is typically between 20° C. and 50° C., and is typically about 25° C. to ensure the proteins are not denatured.

Removal of the oxidized impurity from the reaction mixture may also be run at an elevated temperature (e.g. a temperature of about 30° C. to about 45° C., specifically a temperature of about 37° C.). This can be achieved by heating the stationary phase with an apparatus that maintains an elevated temperature. For example, the reaction may be heated to about 37° C. prior to addition to the stationary phase. This method typically speeds up the reactivity of the reaction increasing capture efficacy of the oxidized impurity.

A modified binding buffer may be used to aid in the removal of the oxidized impurity. The binding buffer will incorporate metals in the form of ions. The stationary phase may be pre-equilibrated with the binding buffer. A concentrated version of the binding buffer may also be added to the reaction mixture of protein and oxidized impurity. The ion concentration may typically range from 10 pM to 100 mM. The typical range will be from 10 nM to 100 μM.

The metals in the binding buffer discussed above can include transition metals, lanthanides, and elements from groups 2, 13, 14, and 15 of the periodic table (Kobayashi S. and Manabe K. Acc. Chem. Res., 35, 2002, 209-217 and reference there in; Hachiya I. and Kobayashi S. J. Org. Chem., 58, 1993, 6958-6960; Kobayashi S. and Hachiya I. J. Org. Chem., 59, 1994, 3590-3596). More commonly, the following ions may be used Al⁺³, Sc⁺³, Fe⁺², Cu⁺², Zn⁺², Y⁺³, Cd⁺², L⁺³, La⁺³, Ce⁺³, Pr⁺³, Nd⁺³, Sm⁺³, Eu⁺³, Gd⁺³, Tb⁺³, Dy⁺³, Ho⁺³, Er⁺³, Tm⁺³, Yb⁺³, Lu⁺³. The anionic counter ions for the above ions can be trifluoromethanesulfonate, chloride, tris(dodecylsulfate), nitrate, nitrite, sulfate, and sulfite, for example.

The invention will now be illustrated by the following non-limiting Examples.

### EXAMPLE 1

### Endotoxin Removal LAL Test

All reagents that will be used during this experiment are listed below. Sodium periodate from Sigma-Aldrich (Cat #311448), endotoxin from Sigma-Aldrich (Cat # L2630), ultra link hydrazied resin from Pierce (PI53149), and LAL test kit from Lonza N283-06 PYROGENT Plus.

A solution of endotoxin at a concentration of 5 EU, 500 μL, in 1×PBS pH 7.4 will be treated with 10 μL of a 10 mM sodium periodate in deionized H₂O. This will be allowed to react for 30 minutes. This will then be purified on a column containing 2 mL of the ultra link hydrazied resin stationary phase. The mixture will be eluted from the column using 1×PBS pH 7.4. The eluted fractions will be tested for endotoxin using a LAL assay.

### EXAMPLE 2

### Endotoxin Removal in the Presence of Protein LAL Test

All reagents that will be used during this experiment are listed below. Sodium periodate from Sigma-Aldrich (Cat #311448), streptavidin produced in E. Coli (Cat # S0677), endotoxin from Sigma-Aldrich(Cat # L2630), ultra link hydrazied resin from Pierce (PI53149), and LAL test kit from Lonza N283-06 PYROGENT Plus.

A solution of endotoxin at a concentration of 5 EU and streptavidin at a concentration of 1 mg/mL, 500 uL, in 1×PBS pH 7.4 will be treated with 10 μL of a 10 mM sodium periodate in deionized H₂O. This will be allowed to react for 30 minutes. This will then be purified on a column containing 2 mL of the ultra link hydrazied resin stationary phase. The mixture will be eluted from the column using 1×PBS pH 7.4. The eluted fractions will be tested for endotoxin using a LAL assay.

### EXAMPLE 3

### Testing of Endotoxin Only Removal

All reagents that will be used during this experiment are listed below. Sodium periodate from Sigma-Aldrich (Cat #311448), endotoxin from Sigma-Aldrich (Cat # L2630), ultra link hydrazied resin from Pierce (PI53149), and LAL test kit from Lonza N283-06 PYROGENT Plus.

Endotoxin was dissolved in 1×PBS pH 7.4 and diluted to 2 EU/mL. A sodium periodate solution of 10 mM was made. The hydrazine beads were washed twice with 500 μL of 1×PBS.

In tube A 500 μL of 1×PBS was placed. Into tube B, 500 μL of the dilute endotoxin solution was placed with 50 μL of 1×PBS. Into tube C, 500 μL of the dilute endotoxin solution was placed with 50 μL of 1×PBS. Into tube D, 500 μL of the dilute endotoxin solution was placed with 50 μL of 10 mM sodium periodate. Into tube E, 500 μL of the dilute endotoxin solution was placed with 50 μL of 10 mM sodium periodate. After an hour reaction tubes C and E were added to 100 μL of hydrazide beads, the other reactions were allowed to sit. These were agitated every 5 minutes to keep the beads suspended in the reaction mixture. After an hour all samples were tested for endotoxin using the LAL test kit from Lonza. The test kit sensitivity was 0.125 EU/mL. Tubes A and E were negative for endotoxin. Tubes B, C, and D were positive for endotoxin.

### EXAMPLE 4

### Testing of Endotoxin in the Presence of Protein Removal

All reagents that will be used during this experiment are listed below. Sodium periodate from Sigma-Aldrich (Cat #311448), endotoxin from Sigma-Aldrich (Cat # L2630), IGF-I from R&D Research (Cat# 291-G1), ultra link hydrazied resin from Pierce (PI53149), and LAL test kit from Lonza N283-06 PYROGENT Plus.

Endotoxin was dissolved in 1×PBS pH 7.4 and diluted to 4 EU/mL. The protein was suspended in 1×PBS pH 7.4 at 0.333 mg/mL. A sodium periodate solution of 10 mM was made. The hydrazine beads were washed twice with 500 μL, of 1×PBS.

In tube A 500 μL of 1×PBS was placed. Into tube B, 250 μL of the dilute endotoxin solution and 250 μL of the IGF-I solution was placed with 50 μL of 1×PBS. Into tube C, 250 μL of the dilute endotoxin solution and 250 μL of the IGF-I solution was placed with 50 μL of 1×PBS. Into tube D, 250 μL of the dilute endotoxin solution and 250 μL of the IGF-I solution was placed with 50 μL of 10 mM sodium periodate. Into tube E, 250 μL of the dilute endotoxin solution and 250 μL of the IGF-I solution was placed with 50 μL of 10 mM sodium periodate. After an hour reaction C and E were added to 100 uL of hydrazide beads, the other reactions were allowed to sit. These were placed on a vortexing machine to keep beads suspended in the mixture. After an hour all samples were tested for endotoxin using the LAL test kit from Lonza. The test kit sensitivity was 0.125 EU/mL. Tubes A and E were negative for endotoxin. Tubes B, C, and D were positive for endotoxin.

### EXAMPLE 5

### Testing the Removal of High Concentration of Endotoxin

All reagents that were used during this experiment are listed below. Sodium periodate from Sigma-Aldrich (Cat #311448), endotoxin from Sigma-Aldrich (Cat # L2630), ultra link hydrazied resin from Pierce (PI53149), and Chromogenic LAL test kit from Lonza QCL-1000 Chromogenic LAL 120 Tests.

Endotoxin was dissolved in 1×PBS pH 7.4 and diluted to 1,000,000 EU/mL. A sodium periodate solution of 10 mM was made. The hydrazine beads were washed twice with 500 μL of 1×PBS.

In tube A 500 μL of 1×PBS was placed. Into tube B, 500 μL of the dilute endotoxin solution was placed with 50 μL of 1×PBS. Into tube C, 500 μL of the dilute endotoxin solution was placed with 50 μL of 1×PBS. Into tube D, 500 μL of the dilute endotoxin solution was placed with 50 μL of 10 mM sodium periodate. Into tube E, 500 μL of the dilute endotoxin solution was placed with 50 μL of 10 mM sodium periodate. After an hour reaction tubes C and E were added to 250 μL of hydrazide beads, the other reactions were allowed to sit. These were agitated by slow vortexing. After an hour the reactions were spun at 1000 rpm and the samples were tested for endotoxin using the colorimetric test kit from Lonza. The test kit sensitivity is from 1 EU/mL to 0.125 EU/mL. Tube A was negative for endotoxin. Tube E showed an exdotoxin level of 0.74 EU/mL. Tubes B, C, and D were positive for endotoxin. These were further diluted 1:1,000,000 and showed endotoxin levels that were 0.92, 0.89, 0.90 EU/mL respectively.

### EXAMPLE 6

### Testing the Extent of Removal of Endotoxin from Solutions

All reagents that were used during this experiment are listed below. Sodium periodate from Sigma-Aldrich (Cat #311448), endotoxin from Sigma-Aldrich (Cat # L2630), ultra link hydrazied resin from Pierce (PI53149), and Chromogenic LAL test kit from Lonza QCL-1000 Chromogenic LAL 120 Tests.

Endotoxin was dissolved in 1×PBS pH 7.4 and diluted to 20,000 EU/mL. A sodium periodate solution of 10 mM was made. The hydrazine beads were washed twice with 500 μL of 1×PBS.

In tube A 500 μL of 1×PBS was placed. Into tube B, 500 μL of the dilute endotoxin solution was placed with 50 μL of 1×PBS. Into tube C, 500 μL of the dilute endotoxin solution was placed with 504 of 1×PBS. Into tube D, 500 μL of the dilute endotoxin solution was placed with 50 μL of 10 mM sodium periodate. Into tube E, 500 μL of the dilute endotoxin solution was placed with 50 μL of 10 mM sodium periodate. After an hour reaction tubes C and E were added to 250 μL of hydrazide beads, the other reactions were allowed to sit. These were agitated by slow vortexing. After an hour the reactions were spun at 1000 rpm and the samples were tested for endotoxin using the colorimetric test kit from Lonza. The test kit sensitivity is from 1 EU/mL to 0.125 EU/mL. Tube A was negative for endotoxin. Tube E showed an exdotoxin level below 0.125 EU/mL. Tubes B, C, and D were positive for endotoxin. These were further diluted 1:20,000 and showed endotoxin levels that were 0.95, 0.92, 0.94 EU/mL respectively.

### EXAMPLE 7

### Testing Protein Activity of After Endotoxin Removal

All reagents that were used during this experiment are listed below. Sodium periodate from Sigma-Aldrich (Cat #311448), endotoxin from Sigma-Aldrich (Cat # L2630), human recombinant B-galactosidase from Abnova (Cat# E801A), ultra link hydrazied resin from Pierce (PI53149), Beta-Galactosidase Enzyme Assay System from Promega (Cat #E2000), and Chromogenic LAL test kit from Lonza QCL-1000 Chromogenic LAL 120 Tests.

Endotoxin was dissolved in 1×PBS pH 7.4 and diluted to 100,000 EU/mL. Beta-galactosidase was suspended in 1×PBS pH 7.4 at 0.200 mg/mL. The measured activity for the beta galactosidase was 300 units/mg. A sodium periodate solution of 10 mM was made. The hydrazine beads were washed twice with 500 μL of 1×PBS.

A 0.1 mg/mL solution of beta-galactosidase with an endotoxin concentration of 1,000 EU/mL was the stock that was used for the following experiments.

In tube A 50 μL of 1×PBS was placed. Into tube B was placed 500 μL of 0.1 mg/mL beta galactosidase solution. Into tube C, 500 μL of the beta galactosidase endotoxin solution was placed with 50 μL of 1×PBS. Into tube D, 500 μL of the dilute endotoxin solution was placed with 50 μL of 1×PBS. Into tube E, 500 μL of the dilute endotoxin solution was placed with 50 μL of 10 mM sodium periodate. Into tube F, 500 μL of the dilute endotoxin solution was placed with 50 μL of 10 mM sodium periodate. After an hour reaction tubes D and F were added to 250 μL of hydrazide beads, the other reactions were allowed to sit. These were agitated by slow vortexing. After an hour the reactions were spun at 1000 rpm and the samples were tested for endotoxin using the colorimetric test kit from Lonza. The endotoxin test kit sensitivity is from 1 EU/mL to 0.125 EU/mL. Tubes A, B, and F were negative for endotoxin. Tube F showed an exdotoxin level below 0.125 EU/mL. Tubes C, D, and E were positive for endotoxin. These were further diluted 1:10,000 and showed endotoxin levels that were 0.89, 0.90, 0.92 EU/mL respectively. Beta galactosidase activity was measured of the above solutions. The beta galactosidase sensitivity is from 1 to 6 milliunits of betagalactosidase. Tube A was negative for beta galactosidase while tubes B thru F were positive with a maxed sensitivity. Samples B thru F were diluted 1:5 and the assay was run again. Activity was 5.4, 5.2, 5.1, 5.2, and 5.2 milliunits for tubes B, C, D, E, and F, respectively.

### EXAMPLE 8

### Testing Endotoxin Removal on a Column at Elevated Temperatures

All reagents that will be used during this experiment are listed below. Sodium periodate from Sigma-Aldrich (Cat #311448), endotoxin from Sigma-Aldrich (Cat # L2630), ultra link hydrazied resin from Pierce (PI53149), and Chromogenic LAL test kit from Lonza QCL-1000 Chromogenic LAL 120 Tests.

Endotoxin will be dissolved in 1×PBS pH 7.4 and diluted to 20,000 EU/mL. A sodium periodate solution of 10 mM will be made. The hydrazine beads, 500 μL, will be washed twice with 1000 μL of 1×PBS. These will be poured into a 2 mL column, five columns will be made.

In tube A 200 μL of 1×PBS will be placed. Into tube B, 200 μL of the dilute endotoxin solution will be placed with 20 μL of 1×PBS. Into tube C, 200 μL of the dilute endotoxin solution will be placed with 20 μL of 1×PBS. Into tube D, 200 μL of the dilute endotoxin solution will be placed with 20 μL of 10 mM sodium periodate. Into tube F, 200 μL of the dilute endotoxin solution will be placed with 20 μL of 10 mM sodium periodate. After an hour reaction tubes will be heated to 37° C. These will then be run individual columns at 37° C. Fractions, 0.5 mL, will be collected from each column. Each fraction will be tested for endotoxin using the endotoxin colorimetric test kit from Lonza.

### EXAMPLE 9

### Testing Endotoxin Removal Using Metal Ions

All reagents that will be used during this experiment are listed below. Sodium periodate from Sigma-Aldrich (Cat #311448), aluminum(III) chloride hexahydrate from Ricca Chemical Company (Cat# RDCA0250), ytterbium(III) trifluoromethanesulfonate from Acros (Cat# 434050010), zinc(II) chloride from Acros (Cat# 1969400050), Iron(II) chloride tetrahydrate from Acros (Cat# 205080050), manganese(II) chloride tetrahydrate from Acros (Cat# 193451000), copper(II) chloride dihydrate from Acros (Cat# 315281000), endotoxin from Sigma-Aldrich(Cat # L2630), ultra link hydrazied resin from Pierce (PI53149), and Chromogenic LAL test kit from Lonza QCL-1000 Chromogenic LAL 120 Tests.

Endotoxin will be dissolved in 1×PBS pH 7.4 and diluted to 1,000 EU/mL. A sodium periodate solution of 10 mM will be made. The hydrazine beads will be washed twice with 1000 μL of 1×PBS. The metals will made to a concentration of 50 mM stock concentration in PBS.

In tube A 200 μL of 1×PBS will be placed with 5 μL of 1×PBS. Into tube B, C, D, E, F, and G will be added 200W, of the dilute endotoxin solution to this will be added 20 μL of the 10 mM sodium periodate solution and the reaction will be allowed to proceed for 1 hour at room temperature protected from light. To each tube will be added a 50 mM solution of aluminum(III) chloride, ytterbium(III) trifluoromethanesulfonate, zinc(II) chloride, Iron(II) chloride tetrahydrate, manganese(II) chloride tetrahydrate, and copper(II) chloride respectively. From each of these reactions a 10 μL aliquots will be taken for a t=0 time measurement and each tube will be added to 200 μL of hydrazide beads. The reactions will be agitated by constant light vortexing. Time points at t=2, 5, 10, 20, 30, and 60 minutes will be taken. Each time point will be evaluated using for endotoxin using the endotoxin colorimetric test kit from Lonza.

### EXAMPLE 10

### Testing of Peptidoglycan Removal

All reagents that will be used during this experiment are listed below. Sodium periodate from Sigma-Aldrich (Cat #311448), peptidoglycan from Sigma-Aldrich (Cat # 69554-10MG-F), ultra link hydrazied resin from Pierce (PI53149), and peptidoglycan test kit from Immunetics product BacTx.

A solution of peptidoglycan at a concentration of 10⁵ CFU/mL, 500 μL, in 1×PBS pH 7.4 will be treated with 10 μL of a 10 mM sodium periodate in deionized H₂O. This will be allowed to react for 30 minutes. This will then be purified on a column containing 2 mL of the ultra link hydrazied resin stationary phase. The mixture will be eluted from the column using 1×PBS pH 7.4. The eluted fractions will be tested for peptidoglycans.

### EXAMPLE 11

### Testing of Peptidoglycan Removal in the Presence of Proteins

All reagents that will be used during this experiment are listed below. Sodium periodate from Sigma-Aldrich (Cat #311448), streptavidin produced in E. Coli (Cat # S0677), peptidoglycan from Sigma-Aldrich (Cat # 69554-10MG-F), ultra link hydrazied resin from Pierce (PI53149), and peptidoglycan test kit from Immunetics product BacTx.

A solution of peptidoglycan at a concentration of 10⁵ CFU/mL and streptavidin at a concentration of 1 mg/mL, 500 uL, in 1×PBS pH 7.4 will be treated with 10 μL of a 10 mM sodium periodate in deionized H₂O. This will be allowed to react for 30 minutes. This will then be purified on a column containing 2 mL of the ultra link hydrazied resin stationary phase. The mixture will be eluted from the column using 1×PBS pH 7.4. The eluted fractions will be tested for peptidoglycans.

### EXAMPLE 12

### Testing of Peptidoglycan Removal

All reagents that will be used during this experiment are listed below. Sodium periodate from Sigma-Aldrich (Cat #311448), peptidoglycan from Sigma-Aldrich(Cat # L2630), ultra link hydrazied resin from Pierce (PI53149), and peptidoglycan test kit from Immunetics product BacTx.

Peptidoglycan will be dissolved in 1×PBS pH 7.4 and diluted to 10⁵ CFU/mL. A sodium periodate solution of 10 mM was made. The hydrazine beads will be washed twice with 500 μL of 1×PBS.

In tube A 500 μL of 1×PBS will be placed. Into tube B, 500 μL, of the dilute peptidoglycan solution will be with 50 μL of 1×PBS. Into tube C, 500 μL of the dilute peptidoglycan solution will be placed with 50 μL of 1×PBS. Into tube D, 500 μL of the dilute peptidoglycan solution will be placed with 50 μL of 10 mM sodium periodate. Into tube E, 500 μL of the dilute peptidoglycan solution will be placed with 50 μL of 10 mM sodium periodate. After an hour reaction tubes C and E will be added to 100 μL of hydrazide beads, the other reactions will be allowed to sit. These will be agitated every 5 minutes to keep the beads suspended in the reaction mixture. After an hour all samples will be tested for peptidoglycan using the BacTx kit from Immunetics.

### EXAMPLE 13

### Testing of Peptidoglycan Removal in the Presence of Proteins (Batch Method)

All reagents that will be used during this experiment are listed below. Sodium periodate from Sigma-Aldrich (Cat #311448), peptidoglycan from Sigma-Aldrich (Cat # L2630), IGF-I from R&D Research (Cat# 291-G1), ultra link hydrazied resin from Pierce (PI53149), and peptidoglycan test kit from Immunetics product BacTx.

Peptidoglycan will be dissolved in 1×PBS pH 7.4 and diluted to 10⁵ CFU/mL. The protein will be suspended in 1×PBS pH 7.4 at 0.333 mg/mL. A sodium periodate solution of 10 mM will be made. The hydrazine beads will be washed twice with 500 μL of 1×PBS.

In tube A 500 μL of 1×PBS will be placed. Into tube B, 250 μL of the dilute peptidoglycan solution and 250 μL of the IGF-I solution will be placed with 50 μL of 1×PBS. Into tube C, 250 μL of the dilute peptidoglycan solution and 250 μL of the IGF-I solution will be placed with 50 μL of 1×PBS. Into tube D, 250 μL of the dilute peptidoglycan solution and 250 μL of the IGF-I solution will be placed with 50 μL of 10 mM sodium periodate. Into tube E, 250 μL of the dilute peptidoglycan solution and 250 μL of the IGF-I solution will be placed with 50 μL of 10 mM sodium periodate. After an hour reaction C and E will be added to 100 uL of hydrazide beads, the other reactions will be allowed to sit. These will be placed on a vortexing machine to keep beads suspended in the mixture. After an hour all samples will be tested for peptidoglycan.

### EXAMPLE 14

### Teichoic and Lipoteichoic Acid Removal

All reagents that will be used during this experiment are listed below. Sodium periodate from Sigma-Aldrich (Cat #311448), teichoic and lipoteichoic acids from Sigma-Aldrich (Cat # L3265), ultra link hydrazied resin from Pierce (PI53149), and teichoic and lipoteichoic acids test kit from antibodies-online LTA assay (Cat# ABIN455722).

A solution of teichoic and lipoteichoic acids at a concentration of 0.5 ng/mL, 500 μL, in 1×PBS pH 7.4 will be treated with 10 μL of a 10 mM sodium periodate in deionized H2O. This will be allowed to react for 30 minutes. This will then be purified on a column containing 2 mL of the ultra link hydrazied resin stationary phase. The mixture will be eluted from the column using 1×PBS pH 7.4. The eluted fractions will be tested for teichoic and lipoteichoic acids using a LTA assay.

### EXAMPLE 15

### Teichoic and Lipoteichoic Acid Removal in the Presence of Proteins

All reagents that will be used during this experiment are listed below. Sodium periodate from Sigma-Aldrich (Cat #311448), streptavidin produced in E. Coli (Cat # S0677), teichoic and lipoteichoic acids from Sigma-Aldrich (Cat # L3265), ultra link hydrazied resin from Pierce (PI53149), and teichoic and lipoteichoic acids test kit from antibodies-online (Cat# ABIN455722).

A solution of teichoic and lipoteichoic acids at a concentration of 0.5 ng/mL and streptavidin at a concentration of 1 mg/mL, 500 uL, in 1×PBS pH 7.4 will be treated with 10 μL of a 10 mM sodium periodate in deionized H₂O. This will be allowed to react for 30 minutes. This will then be purified on a column containing 2 mL of the ultra link hydrazied resin stationary phase. The mixture will be eluted from the column using 1×PBS pH 7.4. The eluted fractions will be tested for teichoic and lipoteichoic acids using a LTA assay.

### EXAMPLE 16

### Testing of Teichoic and Lipoteichoic Acids Removal (Batch Method)

All reagents that will be used during this experiment are listed below. Sodium periodate from Sigma-Aldrich (Cat #311448), teichoic and lipoteichoic acids from Sigma-Aldrich (Cat # L3265), ultra link hydrazied resin from Pierce (PI53149), and teichoic and lipoteichoic acids test kit from antibodies-online (Cat# ABIN455722).

A solution of teichoic and lipoteichoic acids at a concentration of 0.5 ng/mL, 500 μL, will be made. A sodium periodate solution of 10 mM will be made. The hydrazine beads will be washed twice with 500 μL of 1×PBS.

In tube A 500 μL of 1×PBS will be placed. Into tube B, 500 μL of the dilute teichoic and lipoteichoic acids solution will be with 50 μL of 1×PBS. Into tube C, 500 μL of the dilute teichoic and lipoteichoic acids solution will be placed with 50 μL of 1×PBS. Into tube D, 500 μL of the dilute teichoic and lipoteichoic acids solution will be placed with 50 μL of 10 mM sodium periodate. Into tube E, 500 μL of the dilute teichoic and lipoteichoic acids solution will be placed with 50 μL of 10 mM sodium periodate. After an hour reaction tubes C and E will be added to 100 μL of hydrazide beads, the other reactions will be allowed to sit. These will be agitated every 5 minutes to keep the beads suspended in the reaction mixture. After an hour all samples will be tested for teichoic and lipoteichoic acids using the LTA assay.

### EXAMPLE 17

### Testing of Teichoic and Lipoteichoic Acids Removal in the Presence of Protein

All reagents that will be used during this experiment are listed below. Sodium periodate from Sigma-Aldrich (Cat #311448), IGF-I from R&D Research (Cat# 291-G1), teichoic and lipoteichoic acids from Sigma-Aldrich (Cat # L3265), ultra link hydrazied resin from Pierce (PI53149), and teichoic and lipoteichoic acids test kit from antibodies-online (Cat# ABIN455722).

A solution of teichoic and lipoteichoic acids at a concentration of 0.5 ng/mL, 500 μL, will be made. A sodium periodate solution of 10 mM will be made. The hydrazine beads will be washed twice with 500 μL, of 1×PBS. The protein will be suspended in 1×PBS pH 7.4 at 0.333 mg/mL.

In tube A 500 μL of 1×PBS will be placed. Into tube B, 250 μL of the dilute teichoic and lipoteichoic acids solution and 250 μL of the IGF-I solution will be placed with 50 μL of 1×PBS. Into tube C, 250 μL of the dilute teichoic and lipoteichoic acids solution and 250 μL of the IGF-I solution will be placed with 50 μL of 1×PBS. Into tube D, 250 μL of the dilute teichoic and lipoteichoic acids solution and 250 μL of the IGF-I solution will be placed with 50 μL of 10 mM sodium periodate. Into tube E, 250 μL of the dilute teichoic and lipoteichoic acids solution and 250 μL of the IGF-I solution will be placed with 50 μL of 10 mM sodium periodate. After an hour reaction tubes C and E will be added to 100 μL of hydrazide beads, the other reactions will be allowed to sit. These will be agitated every 5 minutes to keep the beads suspended in the reaction mixture. After an hour all samples will be tested for teichoic and lipoteichoic acids using the LTA assay.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method comprising:
contacting a mixture that comprises a protein product and an oxidizable impurity with an oxidizing agent to provide a resulting mixture that comprises an oxidized impurity; and
separating the protein product from the oxidized impurity by contacting the resulting mixture with a material that associates with the oxidized impurity.

2. (canceled)

3. The method of claim 1 wherein the mixture is a solution and wherein the solution comprises water, a (C₁-C₃)alcohol, DMSO, dioxane, dimethoxy ethane, acetonitrile, or DMF, or a mixture thereof.

4. The method of claim 1 wherein the protein product is an enzyme, an antibody, a structural or mechanical protein (e.g. actin or myosin), a protein used for cell adhesion, a protein used for cell signaling, a peptide, or a protein utilized in the cell cycle.

5-7. (canceled)

8. The method of claim 1 wherein the oxidizing agent is a periodate salt or lead tetra acetate.

9. The method of claim 1 wherein the oxidizing agent is sodium periodate, potassium periodate, IBZ, or Dess-Martin peroxidase.

10. The method of claim 1 wherein the oxidized impurity comprises one or more aldehyde groups.

11. The method of claim 1 wherein the material that associates with the oxidized impurity covalently binds with the oxidized impurity.

12. The method of claim 11 wherein the material that covalently bonds with the oxidized impurity comprises a solid material that comprises a plurality of hydrazide groups.

13. The method of claim 12 wherein the hydrazide groups have the formula —NH—NH₂.

14. The method of claim 1 wherein the material can be in the form of a bead, a powder, a gel, a nanoparticle, a fabric, a membrane, or a surface.

15. The method of claim 1 wherein the material is a bead.

16. The method of claim 15 wherein the bead comprises sugar (agarose or dextran), silica, polymer (polyacrylamide), glass, metal (magnetic and non-magnetic), a metal coated silica particle, a silica coated metal particle, a sugar coated metal particle, or a polymer coated metal particle.

17. The method of claim 1 wherein the protein product is separated from the oxidized impurity by passing the solution that comprises the protein product and the oxidized impurity through a column that contains the material that associates with the oxidized impurity.

18. The method of claim 17 wherein the protein product and the solution pass through the column and the oxidized impurity does not.

19. The method of claim 1 wherein the protein product is separated from the oxidized endotoxin by contacting the solution that comprises the protein product and the oxidized impurity with a stationary phase that covalently bonds with the oxidized impurity.

20. The method of claim 19 wherein the oxidized impurity covalently bonds with the stationary phase and the protein product does not.

21. The method of claim 1 wherein the oxidizable impurity is an endotoxin, peptidoglycan, teichoic acid, or lipoteichoic acid.

22-25. (canceled)

26. The method of claim 19 wherein the stationary phase comprises a binding buffer.

27. The method of claim 26 wherein the binding buffer comprises metal ions selected from Al⁺³, Sc⁺³, Fe⁺², Cu⁺², Zn⁺², Y⁺³, Cd⁺², Ln⁺³, La⁺³, Ce⁺³, Pr⁺³, Nd⁺³, Sm⁺³, Eu⁺³, Gd⁺³, Tb⁺³, Dy⁺³, Ho⁺³, Er⁺³, Tm⁺³, Yb⁺³, and Lu⁺³.

28-30. (canceled)
